(19) United States
(12) Patent Application Publication
(10) Pub. No.: US 2004/0229948 A1
(43) Pub. Date: Nov. 18, 2004

(54) Method for preventing hepatic encephalopathic episodes

(76) Inventors: Marshall L. Summar, Brentwood, TN (US); Sharron Ema Gargosky, Slottsdale, AZ (US)

Correspondence Address: William J. McNichol, Jr., Reed Smith LLP, 2500 One Liberty Place, 1650 Market Street, Philadelphia, PA, 19103-7301, US

(21) Appl. No.: 10/725,064

(22) Filed: Dec. 1, 2003

Related U.S. Application Data
(63) Continuation-in-part of application No. 10/122,445, filed on Apr. 12, 2002, now abandoned.

(51) Int. Cl. A61K 31/225; A61K 31/22; A61K 31/192

(52) U.S. Cl. 514/547; 514/546; 514/570

(57) ABSTRACT

A method for preventing an initial hepatic encephalopathic episode in persons at risk for hepatic encephalopathic episodes by administering to the person a therapeutically effective amount of at least one phenyl butyrate compounds or a salt, derivative or metabolite of phenyl butyrate in a pharmaceutically acceptable vehicle.

### CROSS REFERENCES TO RELATED APPLICATIONS

This application is a continuation-in-part of application Ser. No. 10/122,445, filed Apr. 12, 2002, which is incorporated herein by reference.

### BACKGROUND OF THE INVENTION

This invention relates to the treatment or prevention of a class of brain disorders known as chronic hepatic encephalopathy. Hepatic encephalopathy is characterized by a progressive loss of brain and mental function, and is associated with disorders of liver function.

Liver disorders that can be associated with hepatic encephalopathy vary widely in their causation and clinical presentation. Hepatitis, cirrhosis, drug or alcohol abuse, and a variety of other disorders can be associated with hepatic encephalopathy. Hepatic encephalopathies can also result from physical disruption of metabolite delivery to the liver.

The loss of mental function associated with hepatic encephalopathies can be severe. Eventually, patients can lose their ability to carry out ordinary life functions, or even to recognize close relatives. The emotional toll taken by this disorder is heavy, as is the financial burden that it imposes on families and the community.

Phenyl butyrate and its metabolite phenyl acetate are known chemical entities. Sodium phenyl butyrate has been approved for use in the United States to treat disorders of urea cycle metabolism, and is sold under the trademark Buphenyl® for that purpose. It has also been reported that certain of this class of components is effective as an anticancer agent (See, U.S. Pat. No. 6,037,376), and as an anti-viral (See, U.S. Pat. Nos. 5,877,213 and 5,710,178).

There is also a patient population known to be at risk for hepatic encephalopathic episodes, including, without limitation, patients who are awaiting liver transplants, surgical and/or portal hypertension patients. These patients may suffer from the following, including but not limited to, congenital atresia or stenosis, thrombosis of portal vein, thrombosis of splenic vein, cirrhosis (including, but not limited to portal, postnecrotic, biliary, Wilson's disease, and hemochromatosis), acute alcoholic liver disease, congenital hepatic fibrosis, idiopathic portal hypertension (hepatoportal sclerosis), schistosomiasis, Budd-Chlari syndrome, constrictive pericarditis, arterial-portal venous fistula, Banti's syndrome and splenomegaly. Patients may also have surgical radiological shunts (“TIPS” or transjugular intrahepatic portosystemic shunt). TIPS patients also include, without limitation, Ascites patients. See Way, Current Surgical Diagnosis & Treatment (1994), 521.

The following factors may also contribute, without limitation, to encephalopathic episodes for at risk patients: the extent of portal-systemic shunt, depressed liver function, intestinal protein load, intestinal flora, azotemia, constipation, the age of the patient, hypokalemia, alkalosis, diuretics, sedatives, narcotics, tranquilizers, infection, hypoxia, hypoglycemia and myxedema. See Current Surgical Diagnosis & Treatment, 535.

Hepatic encephalopathy has the following proposed nomenclature in the art. Type A is encephalopathy associated with acute liver failure, Type B is encephalopathy associated with portal-systemic bypass and no intrinsic hepatocellular disease, and Type C is encephalopathy associated with cirrhosis and portal hypertension or portal systemic shunts. Type C has three subcategories: Episodic hepatic encephalopathy which may be precipitated, spontaneous or recurrent, Persistent hepatic encephalopathy which may be mild, severe or treatment dependent and Minimal hepatic encephalopathy. See Ferenci et al., Hepatic Encephalopathy—Definition, Nomenclature, Diagnosis, and Quantification: Final Report of the Working Party at the 11^{th} World Congress of Gastroenterology, Vienna, 1998, Hepatology, vol. 35, Nov. 3, 2002.

A person at risk for hepatic encephalopathic episodes is a person who has not suffered any hepatic encephalopathic episodes or has not suffered any hepatic encephalopathic episode for an extended period of time (about 12 weeks or longer), but has a disorder or medical condition which creates a risk of hepatic encephalopathic episodes. A hepatic encephalopathic episode is a clinical condition characterized by the presence of cerebral dysfunction in patients with liver disease or dysfunction with a West Haven Criteria grading of mental status of a Grade I or II.

Hepatic encephalopathy has been divided into separate grades depending on the severity and symptoms in the West Haven Criteria. All grading in this specification refers to the West Haven Criteria. Grade I patients exhibit trivial lack of awareness, euphoria or anxiety, shortened attention span and impaired performance of addition. Grade II patients exhibit lethargy or apathy, minimal disorientation for time or place, subtle personality change, inappropriate behavior and impaired performance of subtraction. Grade III patients exhibit somnolence to semistupor (but responsive to verbal stimuli), confusion and gross disorientation. Grade IV patients are in a coma (unresponsive to verbal or noxious stimuli).

### SUMMARY OF THE INVENTION

According to the present invention, phenyl butyrate compounds, their salts, derivatives and metabolites are used to treat chronic hepatic encephalopathy. Treatment according to this invention can arrest and even reverse the loss of mental function associated with chronic hepatic encephalopathies.

In the practice of this invention, phenyl butyrate compounds, their salts, derivatives and metabolites are administered in an amount effective to achieve an optimum clinical result.

In another embodiment of the invention, phenyl butyrate compounds, their salts, derivatives and/or metabolites are administered to a person at risk of hepatic encephalopathic episodes in amount effective to prevent, minimize (or lessen the severity of), or delay an initial hepatic encephalopathic episode. An initial hepatic encephalopathy episode is the first episode of the patient.

In another embodiment of the invention, phenyl butyrate compounds, their salts, derivatives and/or metabolites are administered to a person at risk of hepatic encephalopathic episodes in amount effective to prevent, minimize (or lessen the severity of), or delay a hepatic encephalopathic episode, after the patient has not had an episode for at least 12 weeks.

Patients with hepatic encephalopathy type A, B or C may have no recognizable clinical symptoms of brain dysfunction. Sometimes patients with grade I hepatic encephalopathy are described as having subclinical hepatic encephalopathy. However, administering phenyl butyrate compounds, their salts, derivatives and/or metabolites to one at risk of an episode before the clinical symptoms appear prevents the episodes or at least lessen the number and/or severity of episodes.

In a prevention embodiment of the invention, the patient has never had an encephalopathic episode.

In another prevention embodiment of the invention, the patient has not had an encephalopathic episode in at least about 12 weeks.

The risk of hepatic encephalopathic episodes for TIPS patients were noted in the following studies. In one study (Sanyal A J, Freedman A M, Shiffman M L, et al., Portosystemic encephalopathy after transjugular intrahepatic portosystemic shunt: results of a prospective controlled study. Hepatology 1994; 20: 46-55, herein incorporated by reference), thirty TIPS patients were followed for 180 days and 9 of these patients experienced 24 episodes of hepatic encephalopathy; 6 of the 9 had a history of hepatic encephalopathy before TIPS and were receiving lactulose after the TIPS procedure. Fourteen of these 24 episodes occurred in the first 30 days after the TIPS procedure.

In another study (Riggio O, Merli M, Pedretti G, et al., Hepatic encephalopathy after transjugular intrahepatic portosystemic shunt. Dig. Dis. Sci. 1996; 41: 578-84, herein incorporated by reference), 15 out of 47 TIPS patients experienced 20 hepatic encephalopathic episodes over a mean 17 month follow-up. Fourteen of the 20 episodes of hepatic encephalopathy occurred during the first 3 months of follow-up.

In a more recent study (Thuluvath P J, Bal J S, Mitchell S, et al. TIPS for management of refractory ascites: response and survival are both unpredictable. Dig. Dis. Sci. 2003; 48: 542-50, herein incorporated by reference), evaluated the use of TIPS in treatment of refractory ascites (effusion and accumulation of serous fluid in the abdominal cavity) in advanced cirrhosis. Mild hepatic encephalopathy was seen in 12% of patients and severe hepatic encephalopathy was seen in 25% immediately after TIPS.

### DETAILED DESCRIPTION OF THE INVENTION

Sodium phenyl butyrate is conveniently available in a commercial preparation known as Buphenyl®, sold by Ucyclid Pharma, of Scottsdale, Ariz. Buphenyl® is prepared for oral delivery in tablet or powder form.

Other related compounds which are useful in the current invention are the salts, derivatives and metabolites of phenyl butyrate. These are well known in the art. For example, phenyl butyrate compounds are defined to include but are not limited to phenyl butyrate, phenyl acetate, sodium benzoate, glyceryl-tri(4 phenyl butyrate), phenylbutyrylglutamines, phenylalkanes, phenylalkenes, and their acids, alcohols, salts, amines, esters, ethers and glycerides, salts, derivatives and metabolites.

U.S. Pat. No. 4,456,942 discloses a group of phenyl acetate derivatives useful in the present invention. These compounds may be described by the following formula:

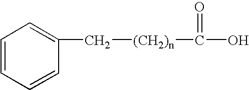

where n is 2, 4, 6 or 8.

Another group of compounds useful in the present invention is disclosed in U.S. Pat. No. 5,968,979, which describes phenylalkanoic esters of glycerol according to the following formula:

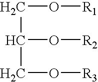

where R₁, R₂ and R₃ are independently, H

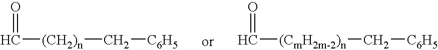

where n is 0 or an even number from 2-24 and m is an even number from 2-24, provided that at least one of R₁, R₂ and R₃ is not H. Glyceryl-tri(4 phenyl butyrate) is an example of such a compound.

Other compounds useful in the method of this invention include phenylacetic acid, its salts (especially sodium salts), halogenated analogs, and alkyl substituted analogs. Specific examples include sodium phenyl acetate and napthyl acetate.

The use of sodium phenyl butyrate to treat chronic hepatic encephalopathy was demonstrated with a group of six patients. Each of these patients suffered from moderate to severe chronic hepatic encephalopathy, and had lost significant mental function as a consequence of the disorder.

The patients in this group suffered from a variety of liver diseases, including Hepatitis C, cirrhosis, and damage caused by drug abuse. At least one patient suffered from a combination of these disorders.

Each patient was given 6 gm/m²/day of sodium phenyl butyrate, divided into three doses. This was done for seven days, during which time the patient's blood chemistry and overall health was monitored and evaluated.

At the end of the seven day regimen, the patients' mental state was reported.

One patient who had suffered significant impairment regained the ability to balance her checkbook, and her family reported a significant improvement in her ability to communicate with others. Another seriously impaired patient regained the ability to drive his car. All patients reported a recovery of mental function, although this benefit was reported to decrease after the use of the drug was terminated.

The improvement in mental function achieved by the method of the present invention has been apparent, as is reported above. Other techniques for measuring improved mental function, such as the PHES score, and auditory nerve conduction studies can be used to demonstrate the effectiveness of this invention.

The dose used in this study proved to be efficacious. However, the dose used in clinical practice will necessarily be adjusted in accordance with the good clinical judgment of the physician. Factors that will be ordinarily considered in this regard include the patient's tolerance for the drug (some of which are known to be difficult to take orally), the severity of the patient's hepatic encephalopathy, the patient's ability to absorb the drug, the patient's total sodium intake, and other factors. Occasionally, it may be necessary to measure the patient's blood levels of sodium phenyl butyrate and/or its metabolites or secondary markers (including but not limited to ammonia) which are known to one of ordinary skill in the art. Such ongoing clinical observation and dosage adjustment are commonplace in good medical practice.

In the above described experiment, the method of this invention was carried out by administering the drug orally. It may be desirable in some circumstances to administer the drug parentally. Some compounds useful in the practice of this invention may be more effective when administered parentally, and others suffer from unpleasant side effects when admitted orally. Intravenous administration is particularly suitable for comatose patients who can be awakened from the comatose state by this method. Sodium phenyl acetate is well suited to parental administration, especially in combination with sodium benzoate. A suitable regimen consists of an initial loading dose and regular additional doses. For example, in infants, a loading dose of about 200-300 mg/kg (preferably about 250 mg/kg) given over 1-2 hours, followed by daily administration of about 200-300 mg/kg (preferably about 250 mg/kg), divided in three, is effective. In adults, a loading and daily dose of about 3.0 to about 8.0 g/m² (preferably about 5 to about 6 g/m²) is effective.

Generally, the orally administered daily dose of sodium phenyl butyrate used in this invention for treatment is between about 3 and about 12 g/m². More commonly, the daily dose will be between about 6 and about 9 g/m².

In a separate embodiment, patients with advanced liver disease who have recently undergone the TIPS procedure and who may or may not be receiving non-absorbable antibiotics and/or lactulose on a chronic basis are given an oral daily dose of Buphenyl® (sodium phenylbutyrate) tablets 500 mg. The patients are equal to or over 18 years of age, have adequate liver function (ALT (alanine aminotransferase) and/or AST (aspartate aminotransferase) not more than 3 times ULN (upper limit of normal), creatinine clearance >50 ml/min, and are not Grade II, III or IV hepatic encephalopathic. Patients are excluded due to the inability to obtain informed consent, pregnancy, a history of congestive heart failure requiring current therapy, any hospitalization in the previous 14 days, enrollment in another experimental protocol in the last 30 days, concomitant gastrointestinal disease, active gastrointestinal bleeding, clinical states manifest by sodium retention and edema, known hypersensitivity to sodium phenylbutyrate, use of probenecid, haloperidol, valproate and (non-topical) corticosteroids and if they are nursing mothers or women of childbearing age without adequate contraception. The Buphenyl® is administered over 12 weeks. Before receiving the Buphenyl®, patients in this target population are believed to have a risk of hepatic encephalopathic episode equal to or exceeding 30% (+/−10%) over a 12-week period. It is believed that this preventative treatment may reduce the risk by 50%, to a risk of about 15%. The clinical outcome is determined by prevention of a hepatic encephalopathic episode. Biochemical amounts are measured in the blood and/or urine by changes of phenyl butyrate and known metabolites, reduction in ammonia concentration, changes in liver enzymes and changes in branched amino acids concentrations. Neurological status and improvement in the quality of life are also be assessed.

Doses for prevention of hepatic encephalopathic episodes may be dependent on the patient's liver function, and may be titrated as is known in the art, like other drugs products are titrated (e.g. human growth hormone). The dose used in clinical practice will necessarily be adjusted in accordance with the good clinical judgment of the physician. Factors that will be ordinarily considered in this regard include the patient's tolerance for the drug (some of which are known to be difficult to take orally), the patient's ability to absorb the drug, the patient's total sodium intake, and other factors. Occasionally, it may be necessary to measure the patient's blood levels of sodium phenyl butyrate. Such ongoing clinical observation and dosage adjustment are commonplace in good medical practice. These doses may range from about 0.1 g/m²/day to about 15 g/m²/day, preferably about 1 g/m²/day to about 8 g/m²/day, more preferably about 3 g/m²/day to about 8 g/m²/day. It may be beneficial to divide these doses into two or three smaller doses daily (totaling to the daily ranges specified). In several embodiments, these doses may be provided parentally, orally and/or intravenously.

It is understood that while the invention has been described in conjunction with the detailed description thereof, that the foregoing description is intended to illustrate and not limit the scope of the invention, which is defined by the scope of the appended claims. Other aspects, advantages, and modifications are evident from a review of the following claims.

I claim:

1. A method of preventing an initial hepatic encephalopathic episode in a person at risk for hepatic encephalopathic episodes comprising administering to the person a therapeutically effective amount of at least one phenyl butyrate compound in a pharmaceutically acceptable vehicle, wherein the compound comprises one or more of the group consisting of phenyl butyrate, phenyl acetate, sodium benzoate, glyceryl-tri(4 phenyl butyrate), phenylbutyrylglutamines, phenylalkanes, phenylalkenes, and their acids, alcohols, salts, amines, esters, ethers, glycerides, salts, derivatives, and metabolites.

2. The method of claim 1, wherein the compound is administered orally.

3. The method of claim 2, wherein the compound is administered in an amount from about 0.1 to about 15 g/m² day.

4. The method of claim 2, wherein the compound is administered in an amount from about 1 to about 8 g/m²/day.

5. The method of claim 2 wherein the compound is administered in an amount from about 3 to about 8 g/m²/day.

6. The method of claims 2, 3, 4 or 5 wherein the compound comprises sodium phenyl butyrate.

7. The method of claims 2, 3, 4 or 5 wherein the compound comprises glyceryl-tri(4 phenyl butyrate).

8. The method of claims 2, 3, 4 or 5 wherein the compound comprises sodium benzoate.

9. The method of claims 2, 3, 4 or 5 wherein the compound comprises sodium phenyl acetate.

10. The method of claims 2, 3, 4 or 5 wherein the compound comprises sodium phenyl acetate and sodium benzoate.

11. The method of claim 1, wherein the compound is delivered parentally.

12. The method of claim 11, wherein the compound is administered in an amount of about 0.1 to about 15 g/m²/day.

13. The method of claim 11, when an initial loading dose of the compound of about 2 to about 13 g/m² is additionally administered to the person.

14. The method of claim 11, wherein the compound is administered in an amount of about 1 to about 8 g/m²/day.

15. The method of claim 11, wherein the compound is administer in an amount of about 3 to about 8 g/m²/day.

16. The method of claims 11, 12, 13, 14 or 15 wherein the compound comprises sodium phenyl butyrate.

17. The method of claims 11, 12, 13, 14 or 15 wherein the compound comprises glyceryl-tri (4 phenyl butyrate).

18. The method of claims 11, 12, 13, 14 or 15 wherein the compound comprises sodium benzoate.

19. The method of claims 11, 12, 13, 14 or 15 wherein the compound comprises sodium phenyl acetate.

20. The method of claim 11, 12, 13, 14 or 15 wherein the compound comprises sodium phenyl acetate and sodium benzoate.

21. A method of lessening severity of an initial hepatic encephalopathic episode in a person at risk for hepatic encephalopathic episodes comprising administering to the person a therapeutically effective amount of at least one phenyl butyrate compound in a pharmaceutically acceptable vehicle, wherein the compound comprises one or more of the group consisting of phenyl butyrate, phenyl acetate, sodium benzoate, glyceryl-tri(4 phenyl butyrate), phenylbutyrylglutamines, phenylalkanes, phenylalkenes, and their acids, alcohols, salts, amines, esters, ethers, glycerides, salts, derivatives, and metabolites and wherein the administering occurs before the hepatic encephalopathic episode.

22. A method of delaying an initial hepatic encephalopathic episode in a person at risk for hepatic encephalopathic episodes comprising administering to the person a therapeutically effective amount of at least one phenyl butyrate compound in a pharmaceutically acceptable vehicle, wherein the compound comprises one or more of the group consisting of phenyl butyrate, phenyl acetate, sodium benzoate, glyceryl-tri(4 phenyl butyrate), phenylbutyrylglutamines, phenylalkanes, phenylalkenes, and their acids, alcohols, salts, amines, esters, ethers, glycerides, salts, derivatives, and metabolites and wherein the administering occurs before the hepatic encephalopathic episode.

23. A method of lessening severity of a hepatic encephalopathic episode in a person at risk for hepatic encephalopathic episodes, wherein at least 12 weeks has passed since the person had a prior episode, comprising administering to the person a therapeutically effective amount of at least one phenyl butyrate compound in a pharmaceutically acceptable vehicle, wherein the compound comprises one or more of the group consisting of phenyl butyrate, phenyl acetate, sodium benzoate, glyceryl-tri(4 phenyl butyrate), phenylbutyrylglutamines, phenylalkanes, phenylalkenes, and their acids, alcohols, salts, amines, esters, ethers, glycerides, salts, derivatives, and metabolites and wherein the administering occurs before the hepatic encephalopathic episode.

24. A method of delaying a hepatic encephalopathic episode in a person at risk for hepatic encephalopathic episodes, wherein at least 12 weeks has passed since the person had a prior episode, comprising administering to the person a therapeutically effective amount of at least one phenyl butyrate compound in a pharmaceutically acceptable vehicle, wherein the compound comprises one or more of the group consisting of phenyl butyrate, phenyl acetate, sodium benzoate, glyceryl-tri(4 phenyl butyrate), phenylbutyrylglutamines, phenylalkanes, phenylalkenes, and their acids, alcohols, salts, amines, esters, ethers, glycerides, salts, derivatives, and metabolites and wherein the administering occurs before the hepatic encephalopathic episode.

25. A method of preventing a hepatic encephalopathic episode in a person at risk for hepatic encephalopathic episodes, wherein at least 12 weeks has passed since the person had a prior episode, comprising administering to the person a therapeutically effective amount of at least one phenyl butyrate compound in a pharmaceutically acceptable vehicle, wherein the compound comprises one or more of the group consisting of phenyl butyrate, phenyl acetate, sodium benzoate, glyceryl-tri(4 phenyl butyrate), phenylbutyrylglutamines, phenylalkanes, phenylalkenes, and their acids, alcohols, salts, amines, esters, ethers, glycerides, salts, derivatives, and metabolites and wherein the administering occurs before the hepatic encephalopathic episode.
